(19) United States
(12) Patent Application Publication
(10) Pub. No.: US 2018/0326375 A1
(43) Pub. Date: Nov. 15, 2018

(54) METHOD OF MAKING A CROSS-LINKED POLYMER GEL AND DEVICE FOR PERFORMING THE METHOD

(71) Applicant: Merz Pharma GmbH & Co. KGaA, Frankfurt am Main (DE)

(72) Inventor: Josef FRIEDRICH, KOELN (DE)

(73) Assignee: Merz Pharma GmbH & Co. KGaA, Frankfurt am Main (DE)

(21) Appl. No.: 15/755,800

(22) PCT Filed: Aug. 31, 2016
(86) PCT No.: PCT/EP2016/001470

§ 371 Date: Feb. 27, 2018

(30) Foreign Application Priority Data
Sep. 1, 2015 (EP) 15002568.2

(51) Int. Cl. B01F 7/30; A61L 27/20; A61L 27/52; C08J 3/24; C08J 3/075; B01D 61/24; B01D 61/28; B01F 7/16; B01F 15/00; B01F 15/02; B01F 7/00

(52) U.S. Cl. CPC B01F 7/30; B01F 2215/0034; A61L 27/52; C08J 3/24; C08J 3/075; B01D 61/243; B01D 61/28; B01F 7/161; B01F 15/00253; B01F 15/0237; B01F 7/00616; A61L 2430/34; B01F 2015/00642; B01F 2215/0481; A61L 27/20

(57) ABSTRACT

The present invention relates to a method of making a composition, the composition comprising a cross-linked polymer and water, comprising at least the following steps: a) providing a first mixture, comprising at least one polymer and an aqueous alkaline solution; b) generating a gel by cross-linking the at least one polymer with a cross-linking agent; c) neutralizing the generated gel by using a neutralization solution; d) optionally adding a second mixture, comprising a cross-linked and/or non-cross-linked polymer and water, to the neutralized gel obtained in step c); e) dialyzing the gel obtained in step d) or dialyzing the gel obtained in step c) and optionally subsequent addition of the second mixture, comprising a cross-linked and/or non-cross-linked polymer and water; and f) filling the dialyzed gel obtained in step e) into a container, wherein at least two or three or four or five or all of steps a) to f) are automated.

### FIELD OF THE INVENTION

The present invention relates to a method of making a composition which comprises a cross-linked polymer, in particular cross-linked hyaluronic acid, and water, and a device for performing the method. Furthermore, the present invention relates to a dialysis cell for use in the device or in the method according to the invention.

### BACKGROUND OF THE INVENTION

Hyaluronic acid is a polymer natural to the human or animal body, which since some time is employed in medicine in different fields such as in orthopedics and in ophthalmology. Nowadays, hyaluronic acid is increasingly used in aesthetic medicine and in plastic surgery. The broad application of hyaluronic acid is particularly due to its very high binding capability of water. In aqueous medium, even at low concentration of hyaluronic acid, viscoelastic gels are formed, which are biologically degradable, and which have advantageous properties.

Pure hyaluronic acid is relatively fastly degraded in the human body. For this reason, hyaluronic acid molecules are often chemically cross-linked with one another, whereby the degradation is considerably decelerated, and the desired effects are maintained for a period of about six to twelve months. Thus, cross-linked hyaluronic acid gels are, among others, frequently used in the aesthetic medicine for the treatment of wrinkles, contouring and volume increase of the upper and lower lip, the improvement of the contours of the cheeks and chin, and for corrections of the nose, etc.

The known hyaluronic acid gels can be roughly classified in two different types, namely mono-phased and bi-phased gels. The mono-phased hyaluronic acid gels consist of a single phase and are non-particulate. The preparation of such mono-phased gels is disclosed, for example, in WO 2008/068297, U.S. Pat. No. 8,450,475, U.S. Pat. No. 8,455,465, U.S. Pat. No. 7,741,476, and U.S. Pat. No. 8,052,990. Known commercial mono-phased hyaluronic acid gels are, for example, Juvéderm®, Teosyal®, Glytone®, and the mono-phased, double-cross-linked hyaluronic acid gels, which are known as Belotero®, Esthélis®, Fortélis® Extra and Modélis® Shape.

The bi-phased hyaluronic acid gels comprise a cross-linked hyaluronic acid material, which is dispersed in a liquid phase. Such gels are particulate and may be made as described in, for example, EP 0 466 300. Commercially available bi-phased gels are, for example, Hylaform®, Restylane®, and Perlane®.

The making of cross-linked hyaluronic acid gels occurs by means of a multi-step process. For example, DE 10 2008 053 892 A1 describes that for the making of a medical implant, initially hyaluronic acid is suspended in NaOH solution, and, after complete dissolution, BDDE (1,4-butanediol diglycidyl ether) is added. After cross-linking, non-reacted BDDE and its degradation products are removed by means of diafiltration against water. Furthermore, DE 35 20 008 C2 discloses that initially a sodium hyaluronate is mixed with a NaOH solution, and subsequently divinyl sulfone (DVS) is admixed. The formed cross-linked gel is then kept without action for one hour, and then it is added to distilled water for swelling overnight. Subsequently, it is broken into small particles by means of vigorous stirring. The particles are filtered off, and are rinsed several times with water, and are then processed to a hyaluronic acid gel.

Methods of making gels based on polysaccharides such as hyaluronic acid are further disclosed in WO 2014/064633, US 2013/210760, US 2013/203696, WO 2010/115081, WO 2012/062775, WO 2013/185934, WO 2009/077399 and WO 2008/034176.

Due to limited flow properties, hyaluronic acid gels are, in general, more difficult to produce and to process at industrial scale compared to products having unlimited flow properties. Thus, the making and processing of hyaluronic acid gels frequently requires a relatively high manual time and effort. This is insofar disadvantageous as quality and reproducibility are decreased and production time and costs are increased.

### OBJECT OF THE INVENTION

The present invention is based on the object to provide an improved method of making a composition comprising a cross-linked polymer, in particular comprising cross-linked hyaluronic acid and water, and an apparatus for performing said method.

### SUMMARY OF THE INVENTION

In a first aspect, the invention relates to a method of making a composition, the composition comprising a cross-linked polymer, in particular cross-linked hyaluronic acid, and water, comprising at least the following steps a) to f):
a) providing a first mixture comprising at least one polymer, in particular hyaluronic acid, and an aqueous alkaline solution;
b) generating a gel by cross-linking the at least one polymer, in particular hyaluronic acid, with a cross-linking agent;
c) neutralizing the generated gel by using a neutralization solution;
d) optionally adding a second mixture comprising a cross-linked and/or non-cross-linked polymer, in particular cross-linked and/or non-cross-linked hyaluronic acid, and water, to the neutralized gel obtained in step c);
e) dialyzing the gel obtained in step d), or dialyzing the gel obtained in step c) and optionally subsequently adding the second mixture comprising a cross-linked and/or non-cross-linked polymer, in particular cross-linked and/or non-cross-linked hyaluronic acid, and water, and
f) filling the dialyzed gel obtained in step e) into a container,
   wherein at least two of steps a) to f) are automated.

In a preferred embodiment, at least three of steps a) to f) or at least four of steps a) to f) or at least five of steps a) to f) or all six of steps a) to f) are automated.

According to the present invention, the provision of the first mixture in step a) may be performed thereby that hyaluronic acid is fed to a first container, and the aqueous alkaline solution is fed from a second container passing a particle filter to the first container, for example by reducing the volume of the second container, or by applying positive pressure. Preferably, an amount of hyaluronic acid fed in step a) and/or a fed amount of the aqueous alkaline solution is determined by means of measurement of the weight of the first and/or the second container, for example by means of weighing means.

Further embodiments of the method according to the invention according to the first aspect of the invention are defined in the appended claims.

In a second aspect, the invention relates to a device for making a composition, the composition comprising a cross-linked polymer, in particular cross-linked hyaluronic acid, and water, comprising:

a first container;
a second container, which is connected to the first container by means of a passage;
at least two stirrers;
an accommodation in the passage, in which a particle filter is arranged;
a dialysis cell which is connected to the first container;
a control equipment for performing a method as defined in the first aspect.

Further embodiments of the device according to the invention according to the second aspect of the invention are defined in the appended claims.

In a third aspect, the invention relates to a dialysis cell for dialyzing a gel, comprising:

a first wall comprising a dialysis membrane, which is arranged on a perforated plate;
an opposing second wall comprising a dialysis membrane, which is arranged on a perforated plate; and
a frame, which is connected to the first wall and to the second wall, and which comprises an inlet opening and outlet opening for filling and discharging the gel;
wherein a distance between the first wall and the second wall amounts to 25 mm at the most, in particular amounts to 10 mm at the most.

Preferably, the frame at least comprises one shiftable piston for reducing the volume in the dialysis cell and/or or comprises a protection layer, which is arranged on a side of the dialysis membrane of the first and/or the second wall, which opposes the perforated plate.

In a fourth aspect, the invention relates to an arrangement of dialysis cells comprising a first dialysis cell as defined in the third aspect, and a second dialysis cell as defined in the third aspect, characterized in that the first and the second dialysis cell have the same volume or have different volumes, and have the same distance between the first and second wall.

### DETAILED DESCRIPTION OF THE INVENTION

The following terms in quotation marks are used in the meaning of the invention.

In a first aspect, the invention relates to a method of making a composition, the composition comprising a cross-linked polymer and water, comprising at least the following steps a) to c) and e) to f), and optionally step d):
a) providing a first mixture comprising at least one polymer and an aqueous alkaline solution;
b) generating a (cross-linked) gel by means of cross-linking the at least one polymer with a cross-linking agent;
c) neutralizing the generated gel by using a neutralization solution;
d) optionally adding a second mixture, comprising a cross-linked and/or non-cross-linked polymer and water to the neutralized gel obtained in step c);
e) dialyzing the gel obtained in step d),
   or dialyzing the gel obtained in step c) and optionally subsequently adding the second mixture, comprising a cross-linked and/or non-cross-linked polymer and water; and
f) filling the dialyzed gel obtained in step e) into a container, preferably into a syringe,
   wherein at least two or three or four or five or all steps of steps a) to f) are automated, and wherein the polymer of the second mixture is equal to or different from the polymer contained in the first mixture, and wherein the polymer of the first and the second mixture is preferably hyaluronic acid.

The term “automated” is used in the meaning of DIN V 19233 and means that the method according to the invention is performed partially or completely without manual work, respectively that the device according to the invention is operated partially or completely without manual work.

The composition prepared in the scope of the present invention may contain further substances such as salts, buffer substances, vitamins (e.g. vitamin E, C, B₆), antioxidant (e.g. ascorbic acid or derivatives thereof, zinc oxide), polyols (e.g. glycerol, mannitol), tri-calcium phosphate particles (e.g., alpha- and beta-tri-calcium phosphate and hydroxyapatite particles), agents (e.g. anesthetics, anti-inflammatory agents, stimulus-reducing agents, vasoconstrictive agents or vasodilatory agents, anticoagulants, humidity-providing substances, immuno-suppressives, antibiotics, etc.) as well as growth factors, peptides or proteins (e.g. neurotoxins). In particular, an anesthetic such as lidocaine may be contained in the composition.

Preferably, the anesthetic is a local anesthetic, such as e.g. lidocaine and/or prilocaine. Preferably, the anesthetic, e.g. lidocaine, is contained in the composition made according to the method of the invention in a concentration of from 0.05 to 5.0 wt.-%, 0.1 to 2.0 wt.-%, 0.1 to 2.0 wt.-%, 0.1 to 1.0 wt.-%, 0.1 to 0.5 wt.-%, 0.1 to 0.4 wt.-%, or from 0.2 to 0.3 wt.-%, based on the total weight of the composition. Preferably, said anesthetic may be added simultaneously or subsequent to step e) and prior to step f).

The further substances, which optionally may be contained in the composition, can be added, as a rule, at any point of time after neutralization step c) up to the filling into a container (e.g. a syringe), respectively terminal sterilization of the container, e.g. a syringe. In case of the anesthetics and other low molecular weight compounds such as ascorbinic acid or zinc oxide, same are preferably added after dialysis step e). This particularly applies to lidocaine.

Step a)

The polymer used in the present invention and provided in step a) is preferably a biologically degradable polymer. The polymer is preferably selected from the group consisting of heparosan, hyaluronan, alginic acid, pectin, gellan, chondroitin, keratin, heparin, cellulose, chitosan, carrageenan, xanthan, and possible suitable salts and derivatives thereof. According to the invention, mixtures of different polymers may also be used. In a preferred embodiment, the polymer is hyaluronan. The term “hyaluronan” is synonymously used with the term “hyaluronic acid”.

Although in the following the present invention is described by means of hyaluronan, i.e. hyaluronic acid, this does not mean that the disclosure is in any way limited to hyaluronic acid. Rather, any polymer may be used in place of hyaluronic acid, in particular one or more of the above-mentioned exemplary polymers. This means that in the following the term “hyaluronic acid” or the like is used as a synonym for the term “polymer”, in particular as a synonym for the above-mentioned polymers.

The term “hyaluronic acid (HA)” as used in the present invention, comprises also salts of hyaluronic acid, in particular sodium, potassium, magnesium, and calcium salts.

The HA of the first mixture preferably has an average molecular weight of from 1.0 to 4.0 MDa, more preferred of from 1.1 to 3.8 MDa, in particular of from 1.2 to 3.6 MDa, 1.2 to 2.8 MDa, or from 1.4 to 3.1 MDa, and in particular preferred of from 1.5 to 3.0 MDa, the average molecular weight being measured by the known static light scattering method. Also a mixture of two or more different HAs having different average molecular weights can be used in the scope of the present invention. For example, a mixture of a first HA having an average molecular of from 0.1 to 2.0 MDa, preferably 1.3 to 1.8 MDa, and a second HA having an average molecular weight of from 1.1 to 4.0 MDa, preferably of from 1.2 to 3.5 MDa, or from 1.5 to 3.0 MDa, may be used. For example, also a mixture of a first HA having an average molecular weight of from 0.5 to 1.5 MDa, in particular of from 1.0 to 1.4 MDa, preferably about 1.2 MDa, and a second HA having an average molecular weight of from 2.5 to 3.5 MDa, in particular of from 2.6 to 3.0 MDa, preferably about 2.8 MDa, may be used. This respectively applies also to the second mixture optionally added in the method of the invention, comprising cross-linked and/or non-cross-linked hyaluronic acid and water.

In one embodiment, in the first mixture provided in step a), a hyaluronic acid having an average molecular weight in the range of from 1.2 to 1.8 MDa is used, whereas in the second mixture provided in optional step d) a hyaluronic acid is used having an average molecular weight in the range of from 2.7 to 3.3 MDa.

In one embodiment, the aqueous alkaline solution used in step a) is an aqueous NaOH solution. In one embodiment, it is a 0.4 to 1.1% NaOH solution, in particular a 0.5 to 1% NaOH solution.

An “aqueous alkaline solution” in the meaning of the present invention is also termed herein as “diluted alkaline solution”.

In one embodiment, in step a), the hyaluronic acid, preferably in the form of fibers or as powder, is provided in a first container. It may be fed to said first container by means of a sealable inlet opening. The diluted alkaline solution is fed from a second container to said first container. In one embodiment, the diluted alkaline solution is fed in particular via a filter such as a particle filter, the filter preferably having a pore size between 0.15 μm and 0.25 μm, in particular having a pore size of 0.2 μm. Said particle filter is accommodated in a passage between the first and the second container. Such particle filter has proved to be of particular advantage since it may remove particles that otherwise may negatively affect the properties of the gel to be prepared. Said diluted alkaline solution is fed from the second container into the first container, in particular by means of a sealable valve, which preferably is arranged at the bottom of the first container and/or by means of a sealable valve, which preferably is arranged at the bottom of the second container, in order to make the first mixture.

In one embodiment, in step a), the interior of the first container has prior or during the feeding of the diluted alkaline solution and/or subsequent to the feeding of the diluted alkaline solution, a temperature between 2° C. and 35° C., or 3° C. and 25° C., in particular between 4° C. and 21° C., whereby a decrease of the temperature to about 4° C. is advantageous for longer dissolution periods. In one embodiment, the interior of the first container is tempered to said temperature, in particular by means of operated or controlled heating and/or cooling of the first container.

In one embodiment, a fed amount of the diluted alkaline solution is determined by means of the measurement of the weight of the first and/or the second container, in particular based on a measured weight increase of the first container and/or weight decrease of the second container. Hereby, step a) can be advantageously performed at least partially in an automated manner.

In one embodiment, the diluted alkaline solution is made in the second container, in particular is provided in the second container. Hereby, the making of the diluted alkaline solution may be integrated into the process. The use of a prepared diluted alkaline solution by a separate step or by an external provider is possible.

In one embodiment, the diluted alkaline solution is fed from the second container into the first container by means of reducing the volume of the second container, in particular by inserting a piston into the second container, or by means of applying pressure, or by means of a pump provided said pump is not a peristaltic pump in order to avoid abrasion. Advantageously, in one embodiment, an abrasion from a supply pump is avoided. The transfer of solutions, in particular of the diluted alkaline solution or neutralizing buffer solution, from the second container into the first container, however, may also be performed by means of pressure application. Thereby, the repeated exchange of extruder pistons and stirring device may be avoided.

In another embodiment, in step a), at first an aqueous alkaline solution is provided in the first container, wherein said aqueous alkaline solution preferably is fed from the second container into the first container via a particle filter as described above. Subsequently to said feeding, hyaluronic acid is directly fed into the first container, e.g. by pouring HA into said first container. This sequence further improves homogeneity of the first mixture.

In a further embodiment, it is also possible to feed hyaluronic acid directly to the first container and to feed an aqueous alkaline solution from the second to the first container, wherein said feeding is performed simultaneously.

In particular in order to achieve a homogeneous distribution of hyaluronic acid, in particular in a first phase of solving hyaluronic acid, in one embodiment, a stirrer is operated in the first container at a higher speed in step a).

Typically, after the dissolution of hyaluronic acid in said aqueous alkaline solution, the first mixture provided in the first container according to step a) is in the form of a gel, i.e. the first mixture has viscoelastic properties.

Step b)

The gel prepared in step b) may consist of the first mixture and the cross-linker, or may furthermore comprise additional ingredients. Due to the cross-linking, the gel prepared in step b) is a cross-linked gel.

In one embodiment, in step b), the cross-linker is fed to the first container, in which the first mixture in step a) has been provided, in particular has been made. As explained in more detail in the following, in one embodiment, the method may be advantageously performed substantially with two containers only, i.e. a first and a second container, and thus may be capsuled against the surrounding, and/or may be (partially) automated or is (partially) automated.

The term “cross-linker” comprises all compounds bearing at least two groups, which are capable of reacting with one or more of the functional groups of the hyaluronic acid, and to cross-link same, for example to react with the hydroxyl groups.

Suitable groups of the cross-linker for cross-linking may be selected from carboxyl, amino, epoxide, halogen, vinyl, isocyanate, or protected isocyanate groups. Exemplary compounds, which may be used as cross-linker, are, for example, 1,4-butanediol diglycidyl ether (BDDE), divinyl sulfone, carbodiimide or 1,4-dichlorobutane. In a preferred embodiment, the cross-linker is BDDE, in particular in the form of an aqueous solution of BDDE.

In one embodiment, prior to the feeding of the cross-linker, the cross-linker is tempered to a temperature, which deviates from an actual temperature in the interior of the first container, respectively a temperature of the first solution by 2 to 8° C. at the most, in particular by 4° C. at the most. Advantageously, this may reduce or even avoid the degradation of cross-linker and dissolved hyaluronic acid and improve the mixing of the first mixture with the cross-linker and/or the subsequent cross-linking.

In another embodiment, the cross-linker is tempered to the same temperature as the dissolved hyaluronic acid.

In one embodiment, after the feeding of the cross-linker, the interior of the first container is tempered, in particular corresponding to a pre-determined profile, to a temperature in the range of from 20° C. to 50° C., in particular in the range of from 24° C. to 36° C. The cross-linking may be advantageously carried out overnight at a temperature of 25 to 28° C. Typically, the gel achieves after 5 to 7 hours an advantageous consistency at a temperature in the range of from 33° C. to 37° C. By means of an upper limit of 40° C., an undesired discoloring of the gel may be reduced, preferably may be avoided, and/or a desired viscoelasticity may be obtained. In one embodiment, the interior of the first container is tempered to this temperature, in particular by means of operated or controlled heating and/or cooling of the first container.

In particular in order to achieve a homogeneous distribution between the first mixture and the cross-linker during the tempering, in particular in a first phase of the tempering, in one embodiment, a stirrer is at least temporarily operated in the first container in step b).

In one embodiment, the stirrer is operated process-depending, i.e. time- and/or temperature-depending, and/or with different speeds. In one embodiment, the stirrer is operated, in particular for a pre-determined period and/or with a higher speed, in order to at first distribute the first mixture and the cross-linker as homogeneously as possible, in particular during the feeding of the cross-linker and/or prior to the tempering.

Additionally, the stirrer is operated during the tempering time-, temperature-, and/or consistency-depending. In particular, the stirrer is operated according to a pre-determined tempering profile and/or a measurement of the actual temperature until the interior of the first container is tempered to a pre-determined temperature, optionally additionally to a pre-determined stopping time, in particular with a reduced speed, in particular by means of operating a gear or a drive by means of which the stirrer is drivable, respectively is driven. In one embodiment, the stirrer is stopped subsequently in step b) while the gel is made by means of cross-linking the hyaluronic acid with the cross-linker at a pre-determined time over a pre-determined period.

The consistency of the cross-linked gel formed in step b) may be determined and controlled by e.g. a puncture resistance test. Such tests are known in the art.

For measuring the temperature, a respective temperature means may be provided in the first container. Such temperature means may be a mono-part or multi-part temperature means for the (direct) measurement of the temperature in the interior of the container, and/or for the (indirect) measurement of the temperature in the interior of the container by means of measuring the temperature of the container.

By means of the stirrer, which works process-depending, i.e. time-, temperature-, and/or consistency-depending, at first a good mixing of the first mixture and the cross-linker may be performed in an advantageous manner. By means of the stirrer, which works with different speeds, in particular by means of operating a gear or drive, the stirring may be advantageously adjusted to the respective process phase, in particular at first the first mixture may be homogenized with a higher stirring speed, and subsequently the cross-linker may be mixed with the same or a comparably lower stirring speed.

By means of the subsequent stopping of the stirrer, which is performed process-depending, in particular time- and/or temperature-depending, the subsequent cross-linking may proceed without negatively affecting the formation and properties of the cross-linked gel.

Step c)

In one embodiment, in step c) the neutralization solution is fed, in particular via a particle filter, from the second container to the first container, in which in step b) the cross-linked gel has been made. Such particle filter has proved to be of particular advantage since it may remove particles that otherwise may negatively affect the properties of the gel to be prepared.

In one embodiment, the neutralization solution is made in the second container, in particular from phosphate buffer and hydrogen chloride (HCl), and is fed from said second container to the first container, from which already the diluted alkaline solution and/or the cross-linker has been fed to the first container. As already mentioned above, by means of using the same second container for the provision, in particular for making the diluted alkaline solution and the neutralization solution, in one embodiment, the method may be advantageously substantially performed with two containers only, a first and a second container, and thus may be capsuled against the surrounding and/or may be (partially) automated, respectively is (partially) automated.

In particular, the first container may be correspondingly the first container in which in step a) the first mixture has been provided, in particular has been made. Hereby, in one embodiment, the second container is cleaned prior to the making of the neutralization solution. In one embodiment, in particular the second container and/or the passage between the first and the second container may be rinsed with washing liquid and/or the filter in the passage may be rinsed or may be changed.

In order to provide the neutralization solution prior to the feeding, said neutralization solution may be stirred in the second container. In particular, in order to at first cut solid gel contained in the first container, and thus to ensure that the neutralization solution reacts as extensively and as soon as possible with the gel and neutralizes an excess of alkaline solution, and/or in order to promote the subsequent neutralization, in one embodiment, in step c), a stirrer is operated at least temporarily in the first container. Accordingly, in one embodiment, the first container as well as the second container comprises a stirrer, respectively.

The term “stirrer” encompasses a shaft comprising one or more stirring tools such as paddles. The term “stirrer system” comprises at least two stirrers. In one embodiment, a stirrer system is used e.g. for the first container. In one embodiment, said stirrer system is an orbital stirrer system as described in more detail below.

In one embodiment, the stirrer is operated process-depending, i.e. in particular time-, and/or consistency-depending, and/or with different working surfaces. In one embodiment, the stirrer is operated, in particular for a pre-determined time, with one working surface. In particular said working surface is a sharp-edged working surface in order to at first cut the gel, and thus to increase the surface thereof, in particular for the neutralization of the cross-linked gel. Additionally, in particular subsequently or alternatively, the stirrer is operated, in particular for a pre-determined time, with one working surface, in particular a curved working surface, in order to mix gel and liquid. The working surfaces may be exchanged, as explained in the following, by means of changing the stirring direction of a stirrer having different working surfaces, which oppose one another.

In one embodiment, the interior of the first container exhibits during the neutralization of the gel by means of the neutralization solution a temperature in the range of from 19° C. to 36° C., in particular of 21° C. In particular step c) may be performed at room temperature.

In one embodiment, a fed amount of neutralization solution is determined by means of the weight measurement of the first and/or the second container, in particular based on a measured weight increase of the first container and/or weight reduction of the second container. Hereby, step c) may be performed advantageously in an at least partially automated manner.

In one embodiment, the neutralization solution is fed from the second container by means of reducing the volume of the second container, in particular by means of inserting a piston into the second container, or also by applying pressure or by means of a pump provided said pump is not a peristaltic pump in order to avoid abrasion. In one embodiment, in this manner, advantageously an abrasion from a supply pump may be avoided.

Step d)

In one embodiment, prior to dialyzing, in optional step d), the neutralized gel obtained in step c) is mixed in a mixing container with a second mixture, which is fed from a further container, comprising cross-linked or non-cross-linked hyaluronic acid and water.

In one embodiment, said mixing container and said further container are different from said first and said second container.

In a preferred embodiment, said mixing container is the first container and said further container is the second container.

In the following, a second mixture of cross-linked hyaluronic acid, optionally non-cross-linked hyaluronic acid, and water is termed as “gel”, “further gel”, or the like. Furthermore, also a second mixture of non-cross-linked hyaluronic acid and water or buffer is in the following also sometimes termed as “non-cross-linked hyaluronic acid”, “solution of a non-cross-linked hyaluronic acid”, or the like.

The mixing container may be in particular the above-discussed first container, in which the first mixture in step a) has been provided, and/or the cross-linking has been performed in step b), and/or the cross-linked gel has been neutralized in step c). The further container correspondingly may be the second container.

In one embodiment, the dialysis cell is connected to the mixing container, i.e. the first container, i.e. the dialysis cell is in fluid communication with said mixing container.

In one embodiment, a fed amount of the non-cross-linked hyaluronic acid and/or the further gel may be determined by measuring the weight of the mixing container and/or the further container.

In one embodiment, during the mixing, a stirrer may be operated at low speed. In one embodiment, the stirrer is operated process-depending, i.e. in particular time- and/or consistency-depending, in particular for a pre-determined time.

In one embodiment, by means of the method and the corresponding device described herein, the mixture comprising cross-linked hyaluronic acid gel may be advantageously made in a sealed, respectively capsuled process. In particular by means of using (only) two containers, which communicate with one another via a passage, preferably which is sealed against the surrounding, the first container (reaction or mixing container) and the second container (provision or further container), the measurement of fed amounts by means of weighing said containers and/or the transport of substances between said containers by means of reducing volumes of containers, in particular changeable reducing of volumes of containers, in particular by inserting a piston in the second container.

In one embodiment, if a second mixture comprising a non-crosslinked polymer is added to the neutralized gel obtained in step c), further crosslinking agent may be added to the resulting mixture.

Step e)

In one embodiment, in step e), the gel is transferred from the first container in which said gel has been neutralized in step c), to a dialysis cell, in particular in order to separate off free cross-linker and/or its degradation products still being contained in said gel, for example BDDE, which has been reduced to diolepoxid or tetraol, and/or neutralized and/or non-neutralized alkaline solution, and/or neutralization solution, and/or for swelling the gel with a buffer solution.

In an alternative embodiment, the gel, which is obtained in step d), which is described in the following, is transferred from the container in which step d) has been performed, i.e. the first container, into a dialysis cell, in particular in order to separate off free cross-linker and/or its degradation products still being contained in said gel, for example BDDE, which has been reduced to diolepoxid or tetraol, and/or non-neutralized alkaline solution, and/or neutralization solution, and/or for swelling the gel with a buffer solution.

Thus, dialysis is performed by filling the gel into a dialysis cell which is connected to the first container.

The term “connected to” is synonymously used with the term “in fluid communication”.

In one embodiment, the gel is transported from the first container into the dialysis cell by reducing the volume in the first container.

In one embodiment, the dialysis cell is arranged or will be arranged in a buffer solution, which may be changed during the dialysis in particular continuously and/or discardingly until the desired extent of dialysis has taken place. The dialysis cell may be in fluid communication with a supply unit comprising buffer solution for the dialysis, and a storage unit for the buffer solution, wherein during the dialysis buffer solution, in particular by means of gravity and/or overpressure and/or actively, in particular by means of a pump or the like, flows from the supply unit to the storage unit, and thereby may e.g. flow around the dialysis cell.

The dialysis cell may be a common dialysis hose having a respective dialysis membrane. Preferably, the dialysis cell is a dialysis cell according to a third aspect discussed in the following of the present invention, which is particularly advantageous for the dialysis.

Step f)

After dialysis, the gel may be transferred back to the first container or to an additional mobile storage container. Prior to the filling into a syringe, the gel is either degassed in the first container, or is transferred to the additional mobile storage container, and is degassed therein. The filling in sterile syringes is performed from the first container or from the mobile storage container.

The filling of the syringes is performed to methods known in the art.

According to a second aspect, the invention relates to a device for making a composition, comprising cross-linked hyaluronic acid and water. The device is provided, respectively designed, for performing a method described herein, or a method according to the invention may be performed with said device, in particularly completely or partially automatedly. The device comprises a first container and a second container, which is connected to the first container by means of a passage, in particular a passage, which is sealed against the surrounding. Furthermore, the first container is connected to a dialysis cell, i.e. the first container is in fluid communication with said dialysis cell.

In one embodiment, the first and/or the second container is/are construed as double-walled container(s), in particular in order to temper its interior and/or to thermally isolate it. For tempering the interior of the double-walled container, the heat exchange jacket, which is defined by means of the double-wall, may communicate with a feed of heat exchange means and discharge of heat exchange means. By means of a feed of heat exchange means of a respective temperature having a corresponding volume flow, which is dissipated after flowing through the heat exchange jacket, the interior of the double-walled container may be heated or cooled targetedly, in particular corresponding to a pre-determined temperature profile. In one embodiment, in the heat exchange jacket, baffle plates for directing the flow and/or for increasing the surface are arranged in order to improve a homogeneous tempering. Additionally or alternatively, the first and/or the second container is/are made from stainless steel. In one embodiment, the first and/or second container has/have a usable volume of at least 12 L, in particular of at least 35 L.

In one embodiment, the device is capsuled, respectively sealed. In one embodiment, in particular the first and/or second container is/are sealable, in particular by means of covers, shiftable pistons, and/or valves. The latter is/are preferably arranged at the bottom of the containers, respectively. In the meaning of the present invention, a capsuled, respectively sealed device is a device in which the interior, in which the manufacturing process is performed and the substances are processed, is only accessible by means of openings, which selectively are sealable by means of covers, valves, filters or the like, which are exclusively provided for said device.

The containers are preferably detachably mounted to a basis such that they may be simply exchanged for the scaling of the amount of gel to be produced and/or for cleaning, sterilization and/or maintenance.

In one embodiment, the device comprises one or more stirrers having a drive, which preferably is capsuled and/or is arranged at an outer surface, respectively on a side of a cover of a container of the device, which opposes the side of the interior of the container, in which the stirrer is arrangeable, in particular is arranged. The use of stirrers comprising a mechanical or a slide ring seal or a magnetic coupling is beneficial in order to avoid contamination of the formed cross-linked gel with particles stemming from abrasion.

In one embodiment, a stirrer is arranged, in particular motor-driven, hydraulically, and/or pneumatically, via a positioning unit. Said positioning unit is preferably arranged at the cover, between a working position in which it is in a usable volume of a container, in order to process the content thereof, and may be positioned in a parking position, in which it is arranged outside of the useable volume, in particular adjacent to the container. In one embodiment, the stirrer is furthermore insertable into, respectively retractable from the container by means of said positioning unit. The stirrer may be telescoped, and/or may be positioned in a distance from the container, in particular perpendicularly thereto. Such arrangement may be provided for mixing the educts provided in the containers.

In one embodiment, the device comprises at least one parking container in which a stirrer is arranged in a parking position when it is not arranged in a useable volume of the first or second container.

In one embodiment, the device comprises a first stirrer for the first container, which is arranged in its working position in the useable volume of the first container in order to process the content thereof. In one embodiment, the first container comprises a stirrer system comprising at least two stirrers or stirring tools, in particular paddles, which, in one embodiment, may be rotated by means of a shared drive or individual drives around a tool axis and/or on a ring orbit or planetary orbit, respectively. Thus, the first stirrer may in particular be construed as a so-called planetary mixer comprising (at least) two stirring paddles, which is known as such, for example, from EP 0 096 136 B1, which additionally is referred to, and the content thereof is explicitly incorporated by reference, and thus needs not to be explained in more detail.

In one embodiment, at least one stirring tool of the first stirrer is selectively drivable with different transmissions by means of a gear having at least two different transmissions, respectively may be coupled with the drive, in particular is rotatable around its tool axis. In one embodiment, additionally or alternatively, at least one stirring tool of the first stirrer is drivable by means of a drive comprising at least two motors, which selectively may be coupled to the stirring tool, or may be driven with different speeds. Hereby, in a simple and/or reliable manner, a stirring rate may be adjusted. In particular, the stirrer may be selectively operated with different rates, in particular process-depending. In one embodiment, a drive can consist of a single motor, in particular an electric motor. In another embodiment, the drive may comprise also two or more motors, in particular electric motors, in particular may consist of said two or more motors, wherein in one embodiment, selectively alternatively different motors may be coupled with the stirrer, respectively the stirring tool, which, in analogy to the operating of a gear, is termed as operating of the drive.

Additionally or alternatively, at least one stirring tool of the first stirrer has different working surfaces. The effect exerted to the medium, e.g. the gel, depends on the rotation direction of the stirrer. In particular, the stirring tool has a first working surface, which, in particular, is sharp-edged, preferably in the form of a knife, and a second working surface, which opposes the first working surface in the rotation direction of the tool around its tool axis, and which is different therefrom, in particular curved, preferably edgeless or cylinder-like such that depending on the rotation direction of the stirring tool, alternatively the first or the second working surface, which is different therefrom, may be moved against a medium to be processed by the tool. By reversing the rotational direction of the tool around its axis of rotation, selectively the first or second working surface is applied to the medium, respectively is forced into said medium.

In one embodiment, the device comprises a second stirrer for the second container, which is arranged in its working position in the usable volume of the second container in order to process the content thereof. In one embodiment, the second container comprises a stirring tool, in particular a paddle. It may particularly be construed as a so-called simple mixer, which is known as such, and thus needs not to be explained here in more detail.

In one embodiment, the first and the second stirrer are arranged in their parking position in respective parking containers which are laterally arranged with respect to the first and the second container, respectively. In another embodiment, the device comprises a first parking container in which the first stirrer will be arranged in its parking position or is arranged in its parking position, when it is not arranged in a useable volume of the first container, and a second parking container, in which the second stirrer will be arranged in its parking position or is arranged in its parking position, when it is not arranged in a useable volume of the second container. By means of the arrangement in a parking container, which preferably is sealable, the method, as already explained, may be performed capsuledly and/or (partially) automatedly. In particular, a contamination of the stirrer in its parking position may be reduced, preferably may be avoided.

In one embodiment, the device comprises a supply unit comprising a buffer solution for preparing the neutralization solution, the non-cross-linked gel, for further dilution of gel and for performing the dialysis, and a storage unit for the (consumed) buffer solution.

In one embodiment, the device comprises a first piston for reducing a volume of the first container, which in particular is motor-driven, hydraulically and/or pneumatically shiftable, and/or a second shiftable piston for reducing a volume of the second container, which in particular is motor-driven, hydraulically and/or pneumatically shiftable. In one embodiment, the piston comprises a filter-protected valve in order to allow the expressed air between gel and piston to escape from the container. As already explained, hereby a contamination with abrasion from a supply pump and/or or a damage or impairment of the gel by means of shear forces may be advantageously avoided.

It is a matter of course that any stirrer being present in the container has to be transferred into its parking position prior to the insertion of the piston.

In one embodiment, the first and/or the second piston can be positioned, in particular motor-driven, hydraulically, and/or pneumatically, via a positioning unit, which preferably is arranged at the cover, between a working position in which it is arranged in a container in order to reduce the volume thereof, and a parking position in which it is arranged outside of the container, in particular is arranged adjacent to the container.

In one embodiment, the first and/or the second piston can be inserted, respectively can be retracted, by means of the positioning unit into, respectively from the container. In particular, the first and/or second piston may be telescoped, and/or may be positioned away from the container, in particular perpendicularly thereto. In one embodiment, additionally or alternatively, the first piston may be arranged in the first container by means of the positioning unit in place of the first stirrer, and/or the second piston may be arranged in the second container by means of the positioning unit in place of the second stirrer.

In one embodiment, the first and the second piston, or also a stirrer and a piston, may be arranged in its/their parking position, e.g. in their respective parking containers. In another embodiment, the device comprises a first piston parking container in which the first piston will be or is arranged in the parking position, when it is not arranged in the first container, and a second piston parking container in which the second piston will be or is arranged in the parking position, when it is not arranged in the second container. By means of the arrangement in a preferably sealable (piston) parking container, the process may be performed, as already discussed, capsuledly and/or (partially) automatedly. In particular a contamination of the piston in its parking position may be reduced, preferably may be avoided.

In one embodiment, the device comprises a weighing means, in particular a weighing cell for weight measurement of at least one of its containers, in particular a weighing means that is tared. Hereby, in one embodiment, a feed to the container, respectively a discharge from the container may be determined.

In one embodiment, the device comprises a control device, in particular a programmable control device, for fully or partially automatedly performing a method which is described herein, in particular at least one of the disclosed steps a) to f). In one embodiment, the control device is connected by a signal to drives of the stirrer, actuators for shifting the pistons, the positioning unit for positioning the stirrer/stirrers and/or pistons, devices for temperature measurement for determining temperatures and controlling in the interior of the container via the facility of heating and cooling, and/or weighing means, in order to control same, respectively to obtain measurement data from same.

In one embodiment, the device comprises a washing means, preferably using filtered water, and/or drying using particle-free air and/or a sterilization means, preferably pure steam, for rinsing the first and/or second container and/or the passage and/or the parking units with a washing and/or sterilization fluid. In one embodiment, the sterilization means comprises a supply unit comprising sterilization fluid, and a storage unit for sterilization fluid. During the process, the first and/or second container and/or the passage and/or the parking units may be rinsed with a washing fluid, if necessary. Subsequent to the process, the first and/or second container and/or the passage and/or the parking units are rinsed or cleaned by means of a sterilization fluid.

In a third aspect, the invention relates to a dialysis cell for dialyzing a gel, in particular a dialysis cell for a device described herein, respectively for such a device. The dialysis cell comprises a first wall comprising a dialysis membrane, which is arranged on a perforated plate such to face the inner side, respectively the interior of the cell, or is arranged on a perforated plate such to be on the exterior, respectively opposes an interior of the cell, respectively faces away, a second wall comprising a dialysis membrane opposing the first wall, which is arranged on a perforated plate on the inner or outer side, and a frame, which is connected to the first and second wall, and an inlet opening for filling the gel, and an outlet opening for discharging the gel.

In one embodiment, the frame is detachably connected to the first and second wall, in particular by means of screws or clamps such that walls, in particular the parts thereof, and frame may be exchanged.

In one embodiment, the dialysis membrane is a semi-permeable membrane, in particular made from cellulose, in particular regenerated cellulose, cellulose acetate, cellulose ester, stainless steel, polyamide such as nylon, polyethylene (PE), polypropylene (PP), or the like. In one embodiment, the dialysis membrane has a cut-off volume of 1·10³ Da at the most, in particular 1·10⁴ Da at the most, and/or at least 1·10⁵ Da, and/or a pore size of 30 μm at the most, in particular 25 μm at the most, and/or at least 10 μm. In one embodiment, the dialysis membrane is in the form of a net.

The perforated plate preferably is made from stainless steel or plastic such as polyvinylidene fluoride (PVDF). In one embodiment, the plate has a wall thickness of at least 0.5 mm and/or 4 mm at the most. In one embodiment, the holes of the perforated plate have a diameter between 5 and 10 mm, in particular between 6 and 8 mm, and/or a hole distance between 1 and 5 mm, in particular between 1 and 2 mm. For example, the holes may be round, square or hexagonal breakthroughs. By means of the perforated plates of the first and second wall, a swelling of the gel in the dialysis cell may be advantageously restricted. Additionally or alternatively, a wall that is stabilized by means of the perforated plate is advantageous in view of a flowing around with dialysis solution, respectively dialysis buffer and/or an exchange of the gel in the interior of the cell when performing dialysis. Said wall may function as the rear wall of the dialysis cell.

According to the invention, a distance between the first and the second wall, which can be a minimal, a maximum and/or mean distance, is 25 mm at the most, in particularly 16 mm at the most, preferably 11 mm at the most, and particularly preferred 10 mm at the most. Said distance may be selected in dependence on the viscosity of the gel. Hereby, the maximal diffusion path from the gel to the dialysis membrane, respectively the buffer solution surrounding said dialysis membrane at the outer side, is reduced to 8 mm, respectively 5.5 mm, respectively 5 mm at the most.

In one embodiment, the frame comprises one piston for reducing a volume of the dialysis cell, wherein preferably the piston is shiftable in the opposite direction or transversely to one another. Hereby, gel may be displaced from the dialysis cell.

Additionally or alternatively, the frame comprises a protection layer, which is arranged on a side of the dialysis membrane of the first and/or second wall, which opposes the perforated plate. In particular, if the protection layer is arranged on a side of the dialysis membrane, which faces the inner side, respectively an interior of the cell, the protection layer may protect the dialysis membrane against abrasion from a shiftable piston and/or gel, which is transported through the cell. Additionally or alternatively, the protection layer may fix the dialysis membrane together with the opposing perforated plate, preferably in a form-fit and frictionally engaged manner. The protection layer may in particular be formed by a further plate in the form of a frame, which preferably is made from stainless steel or a plastic such as PVDF. In one embodiment, it has a wall thickness of at least 0.5 mm and/or 2 mm at the most. In one embodiment, the holes of the protection layer have a diameter between 5 and 10 mm, in particular between 6 and 8 mm, and/or a hole distance between 1 and 5 mm, in particular between 1 and 2 mm.

In one embodiment, the dialysis cell comprises a cover, which in particular is frame-like, which is arranged on the outer side on the first and second wall, and, which in particular is detachably connected to the frame, preferably by means of screws and/or clamps, in order to connect the first and the second wall and the frame in a form-fit and/or frictionally engaged manner to one another. By means of this, the dialysis cell may be simply and/or modularly produced and/or maintained. The frame-like cover has a recess by means of which the interior of the cell communicates through the first and second wall with the surrounding buffer solution, and protects the edges of the first and second wall.

In a fourth aspect, the invention relates to a dialysis cell arrangement, in particular suitable for a device described herein. The dialysis cell arrangement comprises a first of the dialysis cells described herein, and a second of the dialysis cells described herein. The first and second dialysis cell have the same or different volumes, in particular same or different wall lengths of the frames thereof, respectively surfaces which may be flown through, of their first and second walls, however have the same distance between their first and second walls. In one embodiment, this allows to selectively use the same or different dialysis cells having the same or different volumes, however, wherein always the maximal diffusion path from the gel to the dialysis membrane remains sufficiently limited. In this manner, the device may be simply scaled by exchanging the first and second dialysis cell.

Further features and advantages result from the dependent claims and the examples. The figures, partially schematized, show in

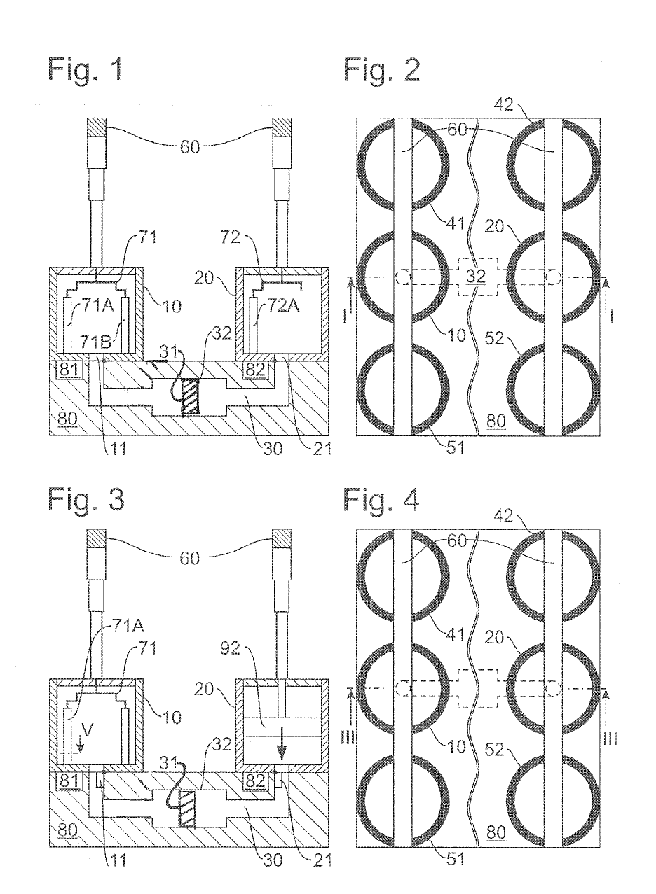

FIG. 1: a sectional view of a device according to one embodiment of the present invention along the line I-I in FIG. 2;

FIG. 2: a top view on the device, wherein stirrers are arranged in a working position;

FIG. 3: a sectional view of the device along the line III-III in FIG. 4;

FIG. 4: a top view on the device, wherein a second piston is arranged in a working position in place of a second stirrer;

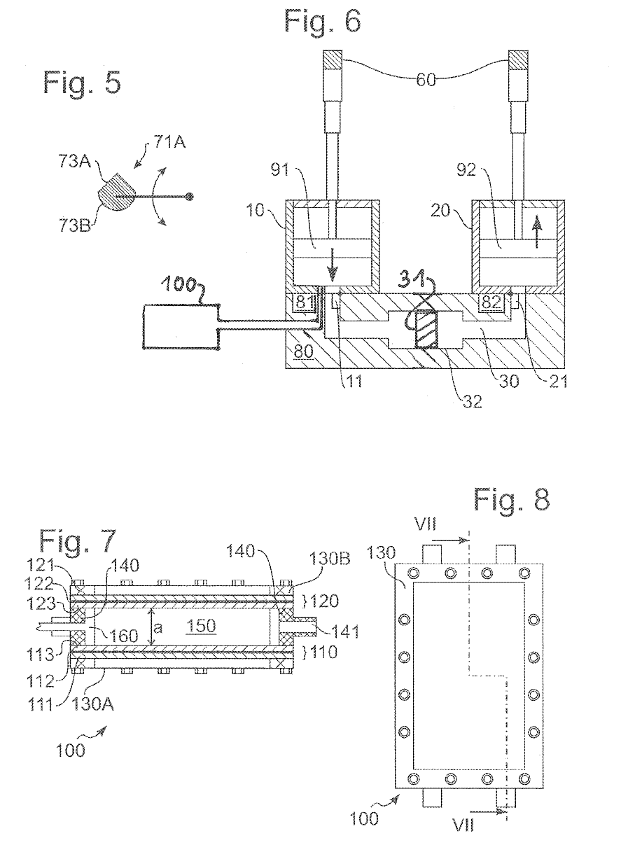

FIG. 5: a sectional view through a stirrer paddle of a first stirrer of the device along line V in FIG. 3;

FIG. 6: the device according to the invention corresponding to the sectional view in FIG. 1, FIG. 3 during dialysis;

FIG. 7: a sectional view of a dialysis cell according to one embodiment of the present invention of the dialysis cell along line VII-VII in FIG. 8; and

FIG. 8: a top view on the dialysis cell.

FIG. 1 shows a longitudinal sectional view of a device according to an embodiment of the present invention, FIG. 2 shows a top view on the device of FIG. 1.

The device comprises a first stainless steel container 10 (first container) having a double jacket, which has, for example, for a scale of 10 kg gel, an inner diameter of approx. 200 mm and a container height of approx. 700 mm, and thus a total volume of approx. 23.5 L, and a useable, respectively useful volume of approx. 10 L. For a scale of 20 kg, the first container 10 has, for example, for the same container height of approx. 700 mm, an internal diameter of approx. 270 mm, and thus a total volume of approx. 40 L, and a useable, respectively useful volume of approx. 20 L, for a scale of 30 kg, for example, for the same height of approx. 700 mm, an inner diameter of approx. 330 mm, and thus a total volume of approx. 60 L, and a useable, respectively useful volume of approx. 30 L. The first container 10 comprises on one side an inspection glass having light, an outlet at the bottom comprising an inlet and outlet valve 11, an inlet for HA powder (not shown), an inlet for BDDE solution (not shown), an outlet at the bottom for transfer of gel from the first container to a dialysis unit 100 (not shown), a sampling tube comprising a valve (not shown), and feed and discharge ports and a water conduit in the double jacket comprising baffle plates for a homogeneous tempering of the container (not shown). The internal side of container 10, i.e. the inner surface, is centrically milled out.

Further, the device comprises a second stainless steel container 20 (second container) comprising a double jacket, which has, for example, for a scale of 10 kg an inner diameter of approx. 200 mm, and a container height of approx. 700 mm, and thus a total volume of approx. 15 L, and a useable, respectively useful volume of approx. 10 L. For a scale of 20 kg, the second container 20 has, for example, for the same container height of approx. 700 mm, an inner diameter of approx. 270 mm, and thus a total volume of approx. 25 L, and a useable, respectively useful volume of approx. 20 L, for a scale of 30 kg, for example, for the same container height of approx. 700 mm, an inner diameter of approx. 330 mm, and thus a total volume of approx. 35 L, and a useable, respectively useful volume of approx. 30 L. The second container 20 comprises on one side an inspection glass having light, an outlet at the bottom comprising valve 21, an inlet for NaOH pellets or NaOH solution (not shown) as well as feed and discharge ports for a tempering of the container (not shown). The internal side of container 20, i.e. the inner surface, is also centrically milled out.

The first container 10 and the second container 20 are connected to one another by means of a passage 30, in which a particle filter 31 is arranged, and which are sealedly integrated in a basis 80 against the surroundings. This means that first container 10 and second container 20 are in fluid communication. Particle filter 31 may be arranged or accommodated in an accommodation 32. The dialysis cell 100 is connected to the first container 10 only.

Further, the device may comprise a mobile storage container for storing dialyzed gel (not shown) made from stainless steel, the dimensions of which correspond to those of the first and second container 10, 20, and which comprises an outlet at the bottom comprising a valve.

Furthermore, the device comprises a first and a second parking container 41, 42 for stirrers, and a first and second parking container 51, 52 for pistons 91, 92 made from stainless steel comprising a bottom outlet comprising a valve, which are arranged in a cover positioning unit 60 (vertical in FIG. 2) on both sides adjacent to the first container 10, respectively second container 20. The parking containers 41, 42 have, for example, for a scale of 10 kg, an inner diameter of approx. 240 mm, and a container height of approx. 700 mm, for a scale of 20 kg, for example, at the same container height of approx. 700 mm, an inner diameter of approx. 290 mm, and for a scale of 30 kg, for example, for the same container height of approx. 700 mm, an inner diameter of approx. 350 mm.

The device further comprises a first stirrer 71 in the form of a single or double planetary mixer for the first container 10 comprising a mixing motor (not shown), whose driving direction is reversible, and which is connected to a container cover, and is capsuled or is arranged at the exterior, and two stirring paddles 71A, 71B, which are arranged in the first container 10 in a working position, which is shown in FIG. 1, with a distance from the bottom and the wall of maximally approx. 1 to 3 mm.

The first stirring paddle 71A may be rotated with up to 150 revolutions per minute. The other stirring paddle 71B is coupled to the mixing motor via a gear, which selectively may have a single or a double transmission, respectively selectively may represent a transmission ratio of 1:1 and 2:1 such that said second stirring paddle also may be rotated with up to 150 revolutions per minute, when the gear is operated in the single transmission, and may be rotated with up to 300 revolutions per minute at the same motor rotation speed, when the gear is operated in the double transmission. The first stirrer 71 may be put down into the first container 10 or the first parking container 41 via a telescopic device of the cover positioning unit 60, and may be positioned between said working position and parking position by means of said cover positioning unit 60.

The blades at the backside of the paddles are sharply edged, which facilitates the cutting of cross-linked gel, however are curved at the front side.

FIG. 5 shows for this a sectional view through stirring paddle 71A along line V in FIG. 3. As is recognizable, stirring paddle 71A preferably has a knife-like first working surface 73A, and an opposing curved second working surface 73B, which is cylinder-like and different therefrom. By means of a reversal of the rotating direction of stirring paddle 71A around its rotational axis (perpendicular to the plane of projection of FIG. 5), which is indicated in FIG. 5 by means of a double-pointed arrow, the first working surface 73A or the second working surface 73B may be selectively inserted into the medium. Accordingly, the stirring paddle 71A has different working surfaces 73A, 73B depending on the rotational direction.

The device further comprises a second stirrer 72 in the form of a single mixer for the second container 20 having a mixing motor (not shown), which is connected to the cover of the container 20 and is capsuled or is positioned at the exterior. Stirrer 72 has a stirring paddle 72A having a large area, which is arranged in a working position in the second container 20, which is shown in FIG. 1, with a distance from the bottom and wall of approx. 1 to 3 mm. The stirring paddle 72A may be rotated with up to 150 revolutions per minute and may be put down into the second container 20 or the second parking container 42 via a telescopic device of the cover positioning unit 60, and may be positioned between said working position and parking position by means of the cover positioning unit 60.

The device further comprises cooling elements and/or heating elements for tempering the first and second container 10, 20 as well as a vacuum system for degassing gels ready to be filled up in the first container 10 and/or the mobile storage container (not shown).

The first container 10 (reaction container) and the second container 20 (supply container) are exchangeably fixed on basis 80, both respectively flanked on both sides by the respective parking containers 41, 42, respectively piston parking containers 51, 52 for placing and optionally cleaning the stirrers 71, 72 and pistons 91, 92, when said pistons 91, 92 are not needed. The additional devices for tempering and degassing as well as a control may be integrated in the basis 80 or may be provided.

In the following, in one embodiment, by means of FIGS. 1 to 5, the making of a cross-linked hyaluronic acid-containing gel, which is ready to be filled up, is explained in more detail. In one embodiment, this process is partially or fully automatedly controlled by means of a control device (not shown). For this, the control device, in particular the cover positioning unit 60, may control the motors of the stirrers 71, 72, the actuators of the pistons 91, 92, and the valves of the containers 10, 20 which are sealed against the surroundings, in particular the bottom valves 11, 21 and/or the inlet valves at the inlets for HA powder, for BDDE solution, and/or NaOH solution, and may receive data from temperature measuring devices, weighing cells, and the like.

NaOH solution is provided in the second container 20, in which the second stirrer 72 is in its working position, and is subsequently transferred via bottom valve 21 of this container 20, the passage 30 and the bottom valve 11 in the first container 10 into the first container 10. In the passage 30, a filter is arranged in the form of a sterilized candle filter 31 having 0.2 μm pore size. Particle filter 31 may be arranged in an accommodation 32.

Hyaluronic acid (HA) is fed into the first container 10 via a cover opening. The first stirrer 71, which is in its working position in the first container 10, is operated for a short period in order to remove HA from the stirring paddles 72A, 72B. In one embodiment, the addition of the HA amount is controlled by means of weighing cells 81 of basis 80 for weighing the first container 10.

The addition of NaOH solution is controlled via weighing cell 81 of the first container 10. For this, at first the second stirrer 72 is telescoped out from its first working position by means of cover positioning unit 60 in the second container 20, and is positioned via the second parking container 42, and is telescoped in its parking position in said container. Subsequently, by means of cover positioning unit 60, the second piston 92 is analogously telescoped out from its parking position in the second piston parking container 52, is positioned via the second container 20 and is telescoped in its working position in said second container 20. Subsequently, it transfers by insertion into the second container 20 the NaOH solution into the first container 10. This is shown in FIG. 3, 4. Alternatively, the transfer may also be provided by means of pressure application.

In another embodiment, NaOH solution is transferred from the second container 20 into the first container 10 as described above. Subsequently, HA is directly fed to container 10.

By means of high speed stirring, the second stirring tool 71B of the first stirrer 71, HA is mixed with the NaOH solution in the first container. Hereby, the gear is operated in the double transmission. As soon as HA is homogeneously dissolved, BDDE is added under stirring at the same speed of the first stirrer 71. In one embodiment, as soon as this cross-linker is homogeneously distributed in the first solution of hyaluronic acid and NaOH solution, the stirrer is stopped and the first container 10 is heated up to a pre-determined temperature.

Subsequently, the cross-linking occurs without stirring at a pre-determined temperature over a pre-determined time of period.

In parallel, in the second container 20, neutralization solution, respectively buffer, is provided. Hereby, at first, the second container 20 is cleaned and is dried, and the particle filter 31, e.g. in the form of a candle filter, is rinsed. Subsequently, phosphate buffer and HCl are added into second container 20, and this neutralization solution is stirred by means of the second stirrer 72 until homogeneity is achieved. The temperature in second container 20 is in the range of from 10 to 35° C. such as 21° C.

Prior to the neutralization, the first container 10 is tempered to room temperature, for example, approx. 21° C., and the gel is cut with the sharp-edged working surface of the first stirrer 71 at low stirring speed. For this, the gear is operated in the simple transmission at low speed.

The provided neutralization solution is transferred via passage 30 comprising the candle filter 31 into the first container 10. The control of the addition of neutralization buffer is gravimetrically performed by means of weighing cells 81 of the first container 10. Thereby, for stirring the neutralization solution and for the subsequent transfer into the first container 10, as already explained above, at first the second stirrer 72 and, subsequently in place of said second stirrer 72, the second piston 92 is positioned in the second container 20. In order to reduce the volume of the second container 20 for transferring the neutralization solution in the first container 10, the second piston 92 is inserted into the second container 92. Alternatively, the transfer may be provided by pressure application.

The gel is stirred at low speed until the neutralization and the pre-determined swelling degree are achieved.

For carrying out dialysis, the remaining neutralization solution in the second container 20 is discharged, and container 20 is rinsed with filtered water. Dialysis buffer having a volume, which is 10 to 50 times of the gel volume, is provided in a supply unit (not shown), which is in fluid communication with dialysis cell 100 via a sterile candle filter (not shown) having 0.2 p pore size for the sterile filtration of the buffer.

The dialysis cell 100 is connected to the first container 10, i.e. in fluid communication with container 10. Optionally, first of all, dialysis cell 100 is assembled from perforated plates, membranes and frames, as described in the following with reference to FIG. 7.

Additionally, stirrer 71 from the first container 10 is positioned by means of cover positioning unit 60 in its parking position in its first parking container 41. Subsequently the first piston 91 may be positioned analogously by means of the cover positioning unit 60 from its parking position in the first piston parking container 51 in its working position in the first container 10, wherein the space between piston and gel, respectively container bottom, is ventilated.

Now, as presented in FIG. 6, when the ventilation valve is closed (not shown), the first piston 91 is put down to the bottom of the first container 10, and thus the gel is inserted from said container 10 into the dialysis cell 100.

From a supply unit (not shown) comprising dialysis solution, respectively buffer, the buffer containment of dialysis cell 100 is filled, preferably is filled while avoiding bubbles, and a flow from the supply unit to a storage unit for discarded dialysis solution (not shown) is effected while dialysis solution is e.g. flowing around dialysis cell 100. The buffer solution may be changed during the dialysis; in particular continuously and/or discardingly until the desired extent of dialysis has taken place.

After dialysis, the gel is in the dialysis cell 100. Subsequent to dialysis, said dialyzed gel is transferred to the first container 10. In one embodiment, the dialyzed gel is transferred to a mobile storage container (not shown).

In another embodiment, a further viscoelastic gel is added to the dialyzed gel contained in container 10. For this, in the second container 20, a further non-crosslinked viscoelastic gel is provided with or without admixtures. The further viscoelastic gel is controlledly slowly transferred to the first container 10 by means of inserting the second piston 92 into container 20. The quantity control is performed by means of weighing cells 81, respectively 82 of the first container 10, respectively the second container 20. The double-phased gel mixture in the first container 10 is re-stirred. Hereby, as already described above, in turn the first stirrer 71 is positioned in place of the first piston 91 in the first container 10.

Prior to the filling into a syringe, the gel is either degassed in the first container 10, or is transferred to an additional mobile storage container (not shown), and is degassed therein. The filling in sterile syringes is correspondingly performed from the first container 10 or from the mobile storage container.

The device shown in FIG. 6 further comprises a control device (not shown) for performing the method defined in the first aspect.

In one embodiment, thus the method is characterized in that in step a) the hyaluronic acid is fed to the first container 10, and the diluted alkaline solution is fed from a second container 20 to the first container 10, in particular via a filter 31, wherein:
the interior of the first and second container 10, 20 have a temperature in the range of from 3° C. to 50° C., and/or
the diluted alkaline solution is made in the second container 20; and/or
the diluted alkaline solution, prior to the feeding to the first container 10, has a temperature, which deviates from the temperature of the cooling medium in the jacket of the container by 2° C. at the most; and/or
the diluted alkaline solution is fed by means of reducing a container volume of the second container 20, or by means of applying pressure; and/or
the diluted alkaline solution is passed through a particle filter 31; and/or
a fed amount of the diluted alkaline solution is determined by means of weighing means 81 of the first and/or second container 10, 20, and
a provided amount of the hyaluronic acid is fed directly into container 10; and/or
after feeding the hyaluronic acid into the first container 10, a stirrer 71 is operated, in particular time- and/or process-depending and/or with high speed until the hyaluronic acid is completely dissolved, preferably homogeneously dissolved.

In a further embodiment, the method is characterized in that in step a) the first mixture is provided in a first container 10, and in step b) the cross-linker is fed to said first container 10, wherein
the interior of the first container 10 is tempered to a temperature in the range of from 4° C. to 50° C., in particular corresponding to a pre-determined profile;
the cross-linker, prior to the feeding, has a temperature, which deviates from the temperature in the interior of the first container 10 by 2° C. at the most; and/or
after feeding the cross-linker into the first container 10, a stirrer 71 is operated, in particular process-depending and/or with different speeds until cross-linker is homogeneous distributed.

In a further embodiment, the method is characterized in that in step b) the cross-linked gel is made in a first container 10, and in step c) the neutralization solution is fed from a second container 20, in particular via a particle filter 31, wherein
in the first and/or second container 10, 20 a stirrer 71, 72 is/are operated, in particular process-depending; and/or
the interior of the first container 10 has a temperature in the range of from 25° C. to 35° C.; and/or
a sample is taken for performing a puncture test;
the neutralization solution is made in the second container 20; and/or
the neutralization solution is fed by means of reducing a container volume of the second container 20; and/or
the neutralization solution is passed through a particle filter 31; and/or
a fed amount of the neutralization solution is determined by means of weight measurement 81 of the first and/or second container 10, 20;
a sample is taken for pH measurement.

In a further embodiment, the method is characterized in that in step c) the gel is neutralized in first container 10, and is transferred in step e) from said first container 10 into a dialysis cell 100, wherein
the dialysis cell 100 is a dialysis cell as defined in the third aspect, and/or
the dialysis cell 100 comprises a buffer solution, which is changed during dialysis, in particular continuously and/or discardingly; and/or
dialysis time is from 2 to 4 days at a temperature of from 15 to 35° C.
dialyzed gel is transferred back to container 10, which serves as mixing container, where further gel and/or an anesthetic and/or an antioxidant may be added, wherein the formed product may be transferred to a mobile storage container.

In a further embodiment, the method is characterized in that in step e) the dialyzed gel is mixed in a mixing container with further cross-linked hyaluronic acid and/or non-cross-linked hyaluronic acid, wherein
in the mixing container a stirrer is operated at lower speed, in particular process-depending; and/or
from a further container cross-linked hyaluronic acid and/or the non-cross-linked hyaluronic acid gel is fed to the mixing container, in particular by reducing a container volume of the further container;
a fed amount of the non-cross-linked hyaluronic acid and/or of the further gel is determined by weight measurement of the mixing container and/or further container; and/or
the mixture is degassed prior to the filling in the mixing container or a storage container, in particular a mobile storage container.

In one embodiment, the mixing container is the first container 10, and the further container is the second container 20.

In the following, with reference to FIG. 7, dialysis cell 100 is described in more detail.

Dialysis cell 100 comprises a flat square basis material having a first and a second wall 110, 120 comprising dialysis membranes 112, 122 and stabilizing perforated plates 111, 121. Perforated plates 111, 121 and membranes 112, 122 are secured with a frame-like cover 130 in a form-fit and frictionally engaged manner. This assembly is further secured by a frame in the form of a non-perforated plate. An internal gel compartment 150 of the dialysis cell has a volume of approx. 1 to 4 L and is sealed with a frame 140, which has openings 141 for filling and discharging dialysis cell 100.

The maximal diffusion paths over days in the gel are not more than 5 to 7 mm depending of the viscosity of gel. The distance (a) between the first and second wall 110, 120 of dialysis cell 100 thus amounts to 10 mm. The dialysis is performed by means of membranes 112, 122, which preferably are construed from a cellulose acetate membrane, or a net made from nylon, PE, PP, or stainless steel. The membranes have a pore size of 10,000 Da at the most, and the net has a pore size of preferably from 10 μm to 25 μm, which are permeable for the component to be separated off, however are impermeable for the cross-linked gel.

Frame 140, perforated plates 111, 121, membranes 112, 122, and cover 130 of dialysis cell 100 are made from plastics, preferably nylon PA12, or stainless steel.

Dialysis cell 100 in the sectional view of FIG. 7 has from the bottom up to the top the following assembly: The first frame-like cover plate 130A of cover 130 is followed by a first perforated plate 111, a first dialysis membrane 112, which is arranged on said first perforated plate 111 on the inner side, and a protection layer in form of a further plate 113 with recess, which is arranged on said membrane on the inner side, which form the first wall 110, which borders gel compartment 150. On the opposing side thereof, a protection layer in the form of a plate 123 with a recess, a second dialysis membrane 122, which is arranged on second perforated plate 121 on the external side which form the second wall 120, followed by a second frame-like cover plate 130B of cover 130, which is arranged on the second wall 120 on the external side. Dialysis cell, respectively frame 140, further comprises at least one shiftable piston 160 for reducing the volume of dialysis cell 100.

One of the short sides (right in FIG. 7) of frame 141 serves for filling gel to be dialyzed and discharging the dialyzed gel. On the insert of the other short side a piston 160 is arranged. The filling insert serves by means of its opening for the filling of the gel to be dialyzed into dialysis cell 100, i.e. it serves as inlet opening. The same opening serves for the discharging of the dialyzed gel from dialysis cell 100, i.e. it serves as outlet opening. After terminating the dialysis, the gel may be extruded using the inserted piston.

In a not shown embodiment of the dialysis, the total gel is filled into dialysis cell 100 (see e.g. FIG. 6). The buffer containment of the dialysis cell is filled with buffer solution with an approx. 10 to 15 fold excess of buffer.

The dialysis cell can also be provided as a dialysis cell arrangement. In one embodiment, said dialysis arrangement comprises a first dialysis cell (100) and a second dialysis cell (100) as defined above with respect to FIG. 7, wherein said first and second dialysis cell (100) have the same or different volumes, but the same distance (a) between the first and the second wall (110,120).

### REFERENCE NUMERALS

10 first container
11 bottom valve
20 second container
21 bottom valve
30 passage
31 particle filter
32 accommodation for particle filter 31
41 first parking container for first stirrer 71
42 second parking container for second stirrer 72
51 first piston parking container for first piston 91
52 second piston parking container for second piston 92
60 cover positioning unit comprising a telescopic unit
71 first stirrer
71A, 71B stirring tools in the form of paddles
72 second stirrer
72A stirring tool in the form of a paddle
80 basis
81, 82 weighing cell (weighing means)
91 first piston
92 second piston
100 dialysis cell
110 first wall
111 perforated plate
112 dialysis membrane
113 protection layer
120 second wall
121 perforated plate
122 dialysis membrane
123 protection layer
130 cover
130A, 130B cover plate
140 frame
141 inlet opening/outlet opening
150 gel compartment
160 piston

1. Method of making a composition, the composition comprising a cross-linked polymer and water, comprising at least a) to f):
a) providing a first mixture comprising at least one polymer and an aqueous alkaline solution;
b) generating a gel by cross-linking the at least one polymer with a cross-linking agent;
c) neutralizing the generated gel by using a neutralization solution;
d) optionally adding a second mixture comprising a cross-linked and/or non-cross-linked polymer and water, to the neutralized gel obtained in c);
e) dialyzing the gel obtained in d),
or dialyzing the gel obtained in c) and optionally subsequently adding the second mixture comprising a cross-linked and/or non-cross-linked polymer and water; and
f) filling the dialyzed gel obtained in e) into a container,
wherein at least two or three or four or five or all of a) to f) are automated.

2. Method according to claim 1, wherein as polymers of the first and second mixture independently from one another heparosan, hyaluronan, alginic acid, pectin, gellan, chondroitin, keratin, heparin, cellulose, chitosan, carrageenan, xanthan, or salts or derivatives thereof or combinations thereof are used, in particular hyaluronan, optionally hyaluronic acid, and/or that the method comprises the addition of an anesthetic, optionally lidocaine or of an antioxidant, optionally mannitol, or of an anesthetic and an antioxidant.

3. Method according to claim 1, wherein the provision of the first mixture in a) is performed thereby that the at least one polymer, in particular hyaluronic acid, is fed to a first container, and the aqueous alkaline solution is fed from a second container to the first container optionally by reducing a container volume of the second container, or by applying pressure to the second container, wherein a provided amount of the at least one polymer, optionally hyaluronic acid, and/or a fed amount of the aqueous alkaline solution is, optionally, determined by means of weight measurement of the first and/or the second container by means of a weighing means.

4. Method according to claim 3, wherein in a) the aqueous alkaline solution is fed from the second container to the first container via a particle filter.

5. Method according to claim 3, wherein in a) the first mixture is provided in the first container, and in b) the cross-linking agent is fed to the first container, wherein the interior of the first container has a temperature in the range of, optionally, 2° C. to 35° C., and the cross-linking agent, prior to the feeding, has a temperature, which deviates from the temperature in the interior of the first container, optionally, by 2° C. at the most, and/or wherein after the feeding of the cross-linking agent the interior of the first container is, optionally, tempered to a temperature in the range of from 20° C. to 50° C.; or
wherein in b) the gel is generated in first container, and in c) the neutralization solution is fed from second container to the first container, wherein the neutralization solution is fed by reducing a container volume of second container, or by means of applying pressure on second container, and/or a fed amount of the neutralization solution is determined by means of weight measurement of the first and/or second container by means of weighing means.

6. Method according to claim 3, wherein a stirrer is operated in container such that prior or simultaneously or subsequently to the feeding of the neutralization solution from the second container to the first container, optionally prior to the feeding, the gel generated in b) in container is cut, wherein for the cutting said stirrer is used.

7. Method according to claim 6, wherein said stirrer comprises at least one stirring tool comprising at least two different working surfaces, wherein a first working surface comprises an edge configured to allow cutting the gel which is generated in b), and a second working surface, which opposes the first working surface, and which is configured to allow mixing the neutralization solution with the gel which has been cut in b) without further cutting it, optionally wherein the second working surface is edgeless or is cylinder-like.

8. Method according to claim 3, wherein in c) the neutralization solution is fed from the second container to the first container via a particle filter.

9. Method according to claim 3, wherein in c) the gel is neutralized in the first container and is transferred in e) from the first container into a dialysis cell, wherein the dialysis cell comprises a buffer solution, which is exchanged during the dialysis, and/or the gel is, for example, transported into the dialysis cell, which is connected to the first container, wherein the exchange of the buffer solution and/or the transportation of the gel into the dialysis cell is preferably automatedly performed; or
wherein in e) the dialyzed gel is mixed in a mixing container with the second mixture, optionally by means of a stirrer, wherein the second mixture is fed from further container to the mixing container, and/or that a fed amount of the second mixture is determined by means of weight measurement by using a weighing means of the mixing container and/or the further container, optionally wherein said mixing container is the first container and said further container is the second container, optionally wherein in e) the second mixture is dialyzed prior to the addition to the dialyzed gel.

10. Method according to claim 1, wherein said method is performed in a device, the device at least comprising:
a first container;
a second container, which is connected to the first container by means of a passage;
at least two stirrers;
an accommodation in the passage in which a particle filter is arranged;
a dialysis cell which is connected to the first container; and
a control device for performing said method.

11. Device for making a composition, the composition comprising at least one cross-linked polymer and water, wherein the device comprises:
a first container;
a second container, which is connected to the first container by means of a passage;
at least two stirrers;
an accommodation in the passage in which a particle filter is arranged;
a dialysis cell which is connected to the first container; and
a control device for performing a method according to claim 1,
wherein the at least one polymer is, optionally, heparosan, hyaluronan, alginic acid, pectin, gellan, chondroitin, keratin, heparin, cellulose, chitosan, carrageenan, xanthan, salts or derivatives thereof or combinations thereof, optionally hyaluronan, optionally hyaluronic acid.

12. Device according to claim 11, comprising a shiftable piston for reducing a volume of the first and/or second container, which is arranged in the first and/or second container; or
comprising a gear comprising at least two different transmissions by means of which stirrer is selectively drivable with different transmissions; and/or characterized by means of a drive comprising at least two motors by means of which stirrer is selectively drivable, and/or comprising means of different working surfaces of a stirring tool of stirrer; and/or comprising means of the rotational direction of the stirrer; or
wherein said stirring tool of stirrer comprises at least two different working surfaces, wherein a first working surface comprises an edge configured to allow cutting the gel generated in b), and a second working surface, which opposes the first working surface, which is configured to allow mixing in c) the neutralization solution with the gel which has been cut in b) without further cutting, optionally wherein the second working surface is edgeless or cylinder-like; or
comprising a weighing means for weight measurement of the first and/or second container; or
comprising a sterilization means for rinsing the first and/or second container and/or the passage with a sterilization fluid; or
characterized by a dialysis cell arrangement, wherein the first or the second dialysis cell of the dialysis cell arrangement is connected to the first container.

13. Dialysis cell for dialyzing a gel, comprising:
a first wall comprising a dialysis membrane, which is arranged on a perforated plate (111);
an opposing second wall comprising a dialysis membrane, which is arranged on a perforated plate; and
a frame, which is connected to the first and second wall, and comprises an inlet and outlet opening for filling and discharging the gel;
wherein a distance (a) between the first and second wall is 25 mm at the most, optionally 10 mm at the most; optionally
wherein the frame comprises at least one shiftable piston for reducing a volume of the dialysis cell, and/or comprising a protection layer, which is arranged on a side of the dialysis membrane opposing inlet and outlet opening.

14. Dialysis cell arrangement comprising a first dialysis cell according to claim 13, and a second dialysis cell according to claim 13, wherein the first and second dialysis cell have the same or different volumes and the same distance (a) between the first and the second wall.

15. Stirrer comprising at least one stirring tool comprising at least two different working surfaces, wherein a first working surface comprises an edge configured to allow cutting, optionally cutting a gel, and a second working surface, which opposes the first working surface, and which is configured to allow mixing without cutting, optionally mixing a gel with water, optionally wherein said second working surface is edgeless or cylinder-like.
